(19) United States
(12) Patent Application Publication
(10) Pub. No.: US 2026/0327241 A1
(43) Pub. Date: Oct. 22, 2026

(54) PROCESS FOR THE PREPARATION OF NALDEMEDINE

(71) Applicant: PROCOS S.P.A., Cameri (IT)

(72) Inventors: Matteo BAUDINO, Rivarolo Canavese (IT); Alberto LENA, Corbetta (IT); Jacopo ROLETTO, Torino (IT); Paolo PAISSONI, Druento (IT)

(21) Appl. No.: 19/484,669

(22) PCT Filed: May 13, 2024
(86) PCT No.: PCT/IB2024/054625

§ 371 Date: Nov. 14, 2025

(30) Foreign Application Priority Data
May 16, 2023 (IT) 102023000009864

(51) Int. Cl. C07D 498/08

(52) U.S. Cl. CPC C07D 498/08

(57) ABSTRACT

The present invention concerns a process for the preparation of naldemedine or a salt thereof, comprising the steps of: a) reacting naltrexone (II) and diethyl pyrocarbonate (DEPC) and treating with abase to give compound (III); b) condensing compound (III) with compound (IV) with formation of the amide bond to obtain naldemedine (I). The process according to the present invention is therefore very simple, effective, and industrially applicable, and above all cost-effective, since it avoids the steps of protection and deprotection of the phenolic group of the structures involved.

### FIELD OF THE INVENTION

The present invention concerns a process for the preparation of naldemedine advantageously comprising a low number of steps.

### STATE OF THE ART

The compound naldemedine is an active ingredient approved by the Food and Drug Administration (FDA) in 2017 and by the European Medicine Agency (EMA) in 2019, and it is used to treat opioid-induced constipation in adults, specifically with chronic non-cancer pain.

Naldemedine acts as a peripherally acting μ-opioid receptor antagonist, and has the following chemical formula (I):

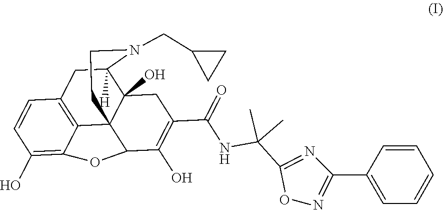 [SMILES: CC(C)(NC(=O)C1=C(O)C2Oc3c(O)ccc4c3[C@@]23CCN(CC2CC2)[C@H](C4)[C@]3(O)C1)c1nc(-c2ccccc2)no1]

and it is marketed as a medicinal specialty in tablets for oral administration under the trade name Rizmoic®.

The known processes for its preparation use, as the starting reagent, Naltrexone having the chemical formula reported in (II):

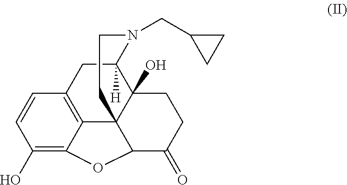 [SMILES: O=C1CC[C@@]2(O)[C@H]3Cc4ccc(O)c5c4[C@@]2(CCN3CC2CC2)C1O5]

Although the synthesis routes reported in the literature show some differences, all current synthetic processes involve the protection of the phenolic group of Naltrexone as the first step.

The protecting group added in the first step is usually removed in the last step to produce the active ingredient naldemedine as a free base, or a salt thereof.

In WO2006126637, the preparation of naldemedine shows a six-step synthesis scheme, which is reported in the following Reaction Scheme 1:

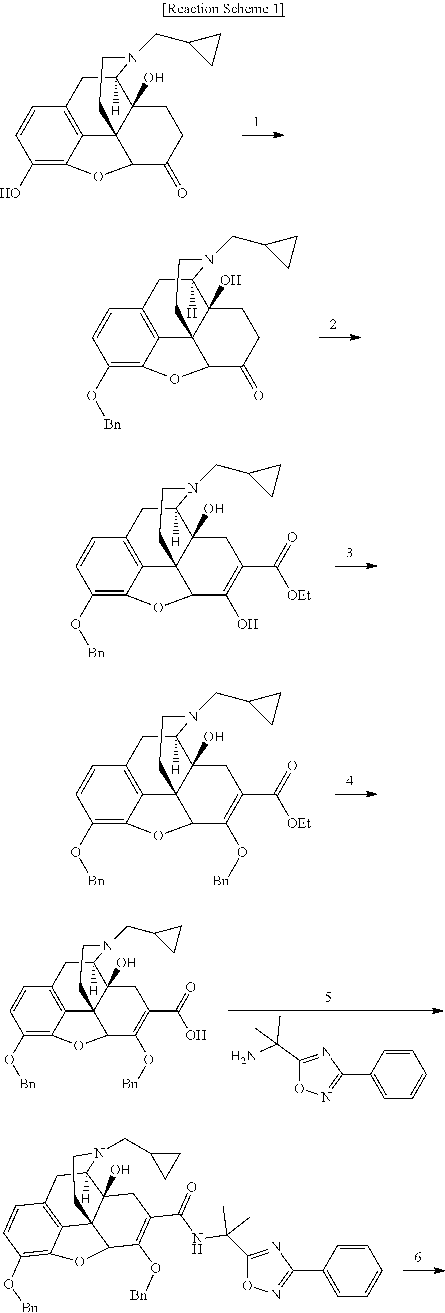

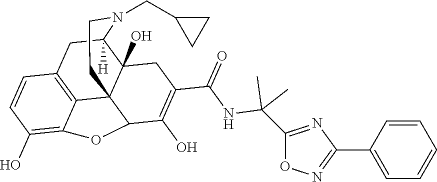 [SMILES: CC(C)(NC(=O)C1=C(O)C2Oc3c(O)ccc4c3[C@@]23CCN(CC2CC2)[C@H](C4)[C@]3(O)C1)c1nc(-c2ccccc2)no1]

As highlighted in Reaction Scheme 1 above, the first and the last synthesis steps are the protection and deprotection of the molecule hydroxyl groups, respectively.

One of the main issues of this synthesis, in addition to the high number of steps, is the obtaining of rather low yields, which makes the synthesis of naldemedine a long and expensive process.

To overcome some of the issues found in this patent application, the same applicant filed a subsequent patent application for an alternative synthesis of naldemedine, published as WO2012063933 and subsequently described in two literature articles (in Org. Process Res. Dev. 2022, 26, 2519-2525 and in Bioorganic & Medicinal Chemistry Letters, 2019, 29, 73-77).

Compared to the process reported in WO2006126637, the synthesis described in WO2012063933 resulted to be optimized for the modification of some of the reaction intermediates, as reported in the following Reaction Scheme 2:

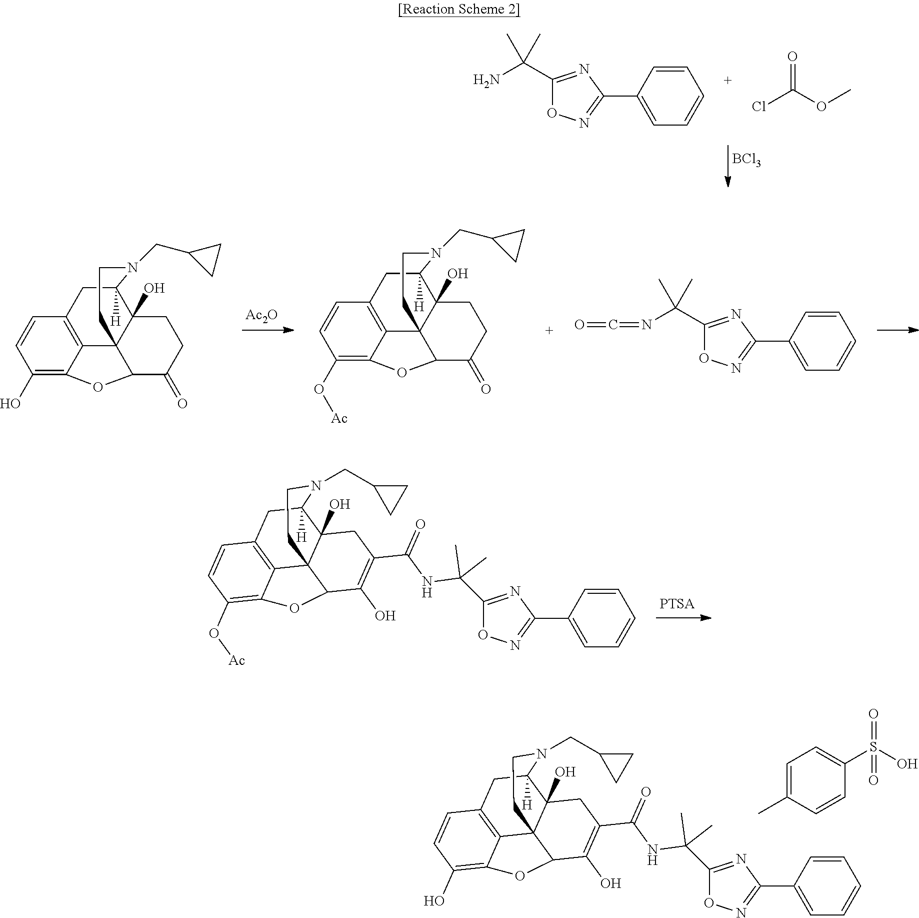

As highlighted in Reaction Scheme 2 above, naldemedine is synthesized through the protection of naltrexone as an acetic ester, condensation with an isocyanate, and finally deprotection of the acetic ester.

An alternative process regarding the synthesis of naldemedine is described in WO2020213911. This application describes a four-step synthesis scheme, namely the protection of naltrexone with benzyl ether, ethoxycarbonylation with diethyl pyrocarbonate (DEPC), amide coupling, and finally the removal of the benzyl group, as reported in Reaction Scheme 3:

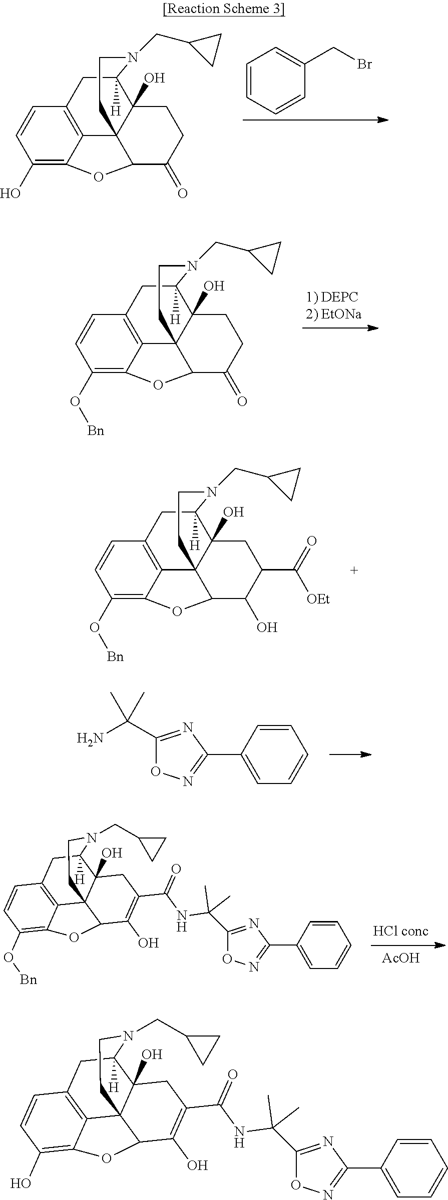

Also in this case, the first and the last steps are the protection and deprotection of the phenolic group.

The object of the present invention is to provide an efficient process for the synthesis of naldemedine, which overcomes the drawbacks of the prior art, and which is at the same time advantageous from an industrial point of view.

### SUMMARY OF THE INVENTION

The object indicated above was achieved through a process that avoids the phenolic group protection and deprotection steps, thus allowing both times and costs of the same process to be reduced.

In a first aspect thereof, the present invention therefore relates to a process for the preparation of naldemedine or a salt thereof, comprising the steps of:
a) reacting naltrexone (II) and diethyl pyrocarbonate (DEPC) and treating with a base to give compound (III)

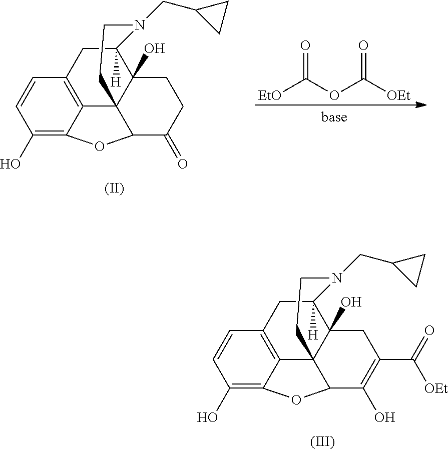

b) condensing compound (III) with compound (IV) with formation of the amide bond to obtain naldemedine (I)

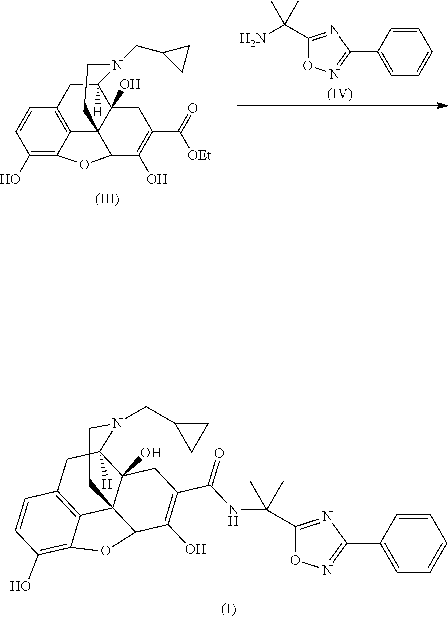

The process according to the present invention is therefore very simple, effective, and industrially applicable, and above all cost-effective.

Therefore, surprisingly, the process of the invention avoids the protection and deprotection steps of the phenolic group of the structures involved, thus allowing to reduce both times and costs of the same process by avoiding the protection and deprotection steps present in the prior art.

Without being tied to any theory, the inventors believe they have managed to avoid the protection and deprotection steps which constitute a technical prejudice based on known processes.

WO2020213911 discloses a process, at page 4, which involves naltrexone, when protected with a benzyl group on the phenolic hydroxyl, to be reacted with diethyl pyrocarbonate replacing the other hydroxyl group of naltrexone which is then ethoxycarbonylated to give a carbonate intermediate as per the following scheme:

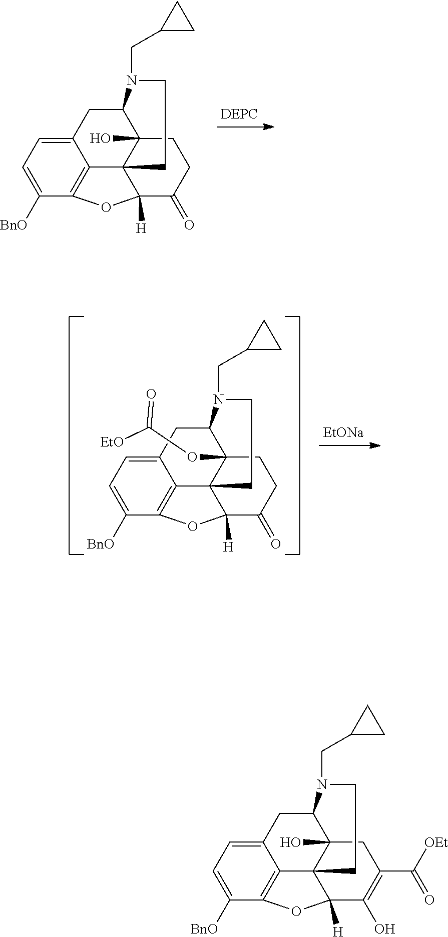

According to WO2020213911, the ethoxycarbonyl group of the carbonate intermediate is transferred to the ketone alpha position by treatment with a base.

The inventors have surprisingly found that step a) of the reaction between unprotected naltrexone (II) and diethyl pyrocarbonate (DEPC) leads to the formation of intermediate (V), and then to the formation of two intermediates (VI) and (VII) according to the scheme:

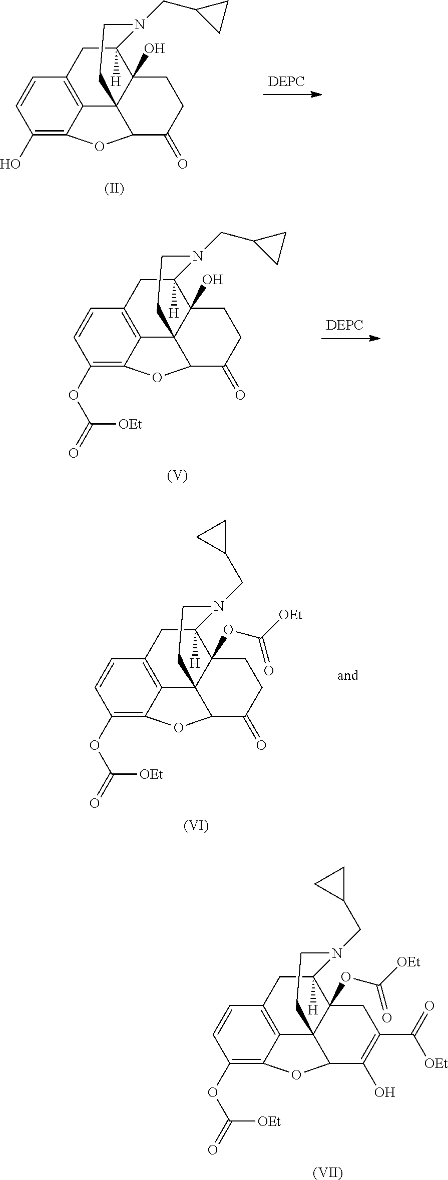

Said intermediates (VI) and (VII), when treated with a base, lead to the direct formation of the compound of formula (III):

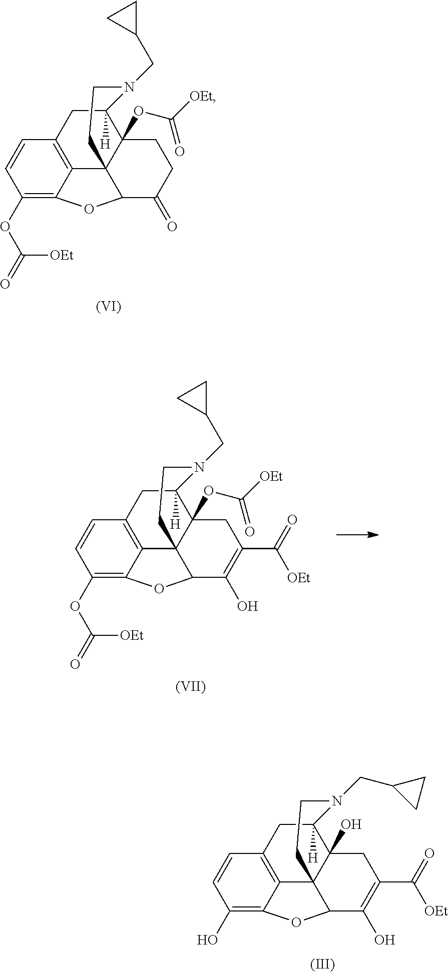

The inventors have thus enabled the provision of a simple process leading to the preparation of naldemedine in an efficient and surprisingly cost-effective manner.

### DESCRIPTION OF THE FIGURES

FIG. 1 reports the UPLC profile of the reaction mixture of step a) showing the peaks relating to Naltrexone (II) and to Naltrexone whose unique phenolic group reacted with DEPC (intermediate V).

FIG. 2 shows the mass spectra of Naltrexone (II) and of Naltrexone whose unique phenolic group reacted with DEPC (intermediate V).

FIG. 3 shows the 1H-NMR spectra of Naltrexone (II) and intermediate (V).

FIG. 4 reports the UPLC profile of the reaction mixture showing the peaks relating to intermediate (VI) and intermediate (VII).

FIG. 5 shows the mass spectra of intermediate (VI) and intermediate (VII).

FIG. 6 shows the 1H-NMR spectrum of the reaction mixture of step a) superimposed with the 1H-NMR spectrum of intermediate (V): the spectrum in darker gray is that relating to intermediate (V), while the spectrum in a lighter shade refers to a 20/80 mixture of intermediates (VI) and (VII).

FIG. 7 shows the exploded area of the spectrum which is highlighted in FIG. 6 by a dotted line.

### DETAILED DESCRIPTION OF THE INVENTION

The present invention relates to a process for the preparation of naldemedine or a salt thereof, comprising the steps of:
a) reacting Naltrexone (II) and diethyl pyrocarbonate (DEPC) and treating with a base to give compound (III):

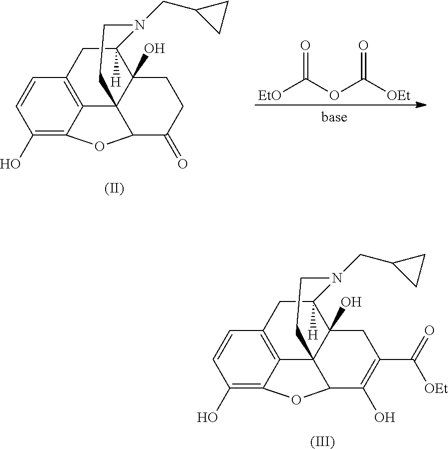

b) condensing compound (III) with compound (IV) with formation of the amide bond to obtain naldemedine (I):

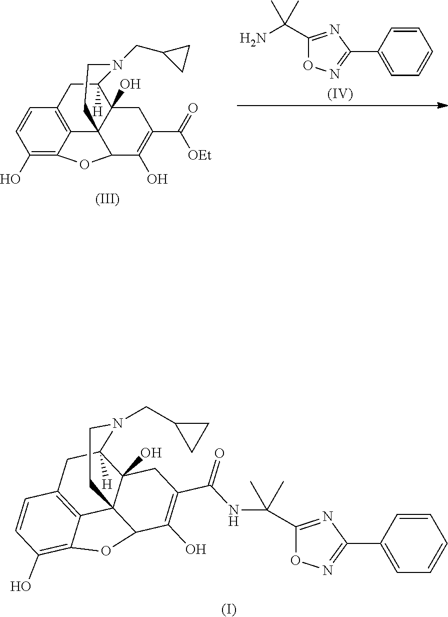

Within the scope of this description and in the subsequent claims, all numerical quantities indicating amounts, parameters, percentages, and so on, are to be understood as preceded in all circumstances by the term “about” unless otherwise indicated.

According to the invention, step a) involves reacting naltrexone (II) and diethyl pyrocarbonate (DEPC) and treating with a base to give compound (III).

Advantageously, step a) comprises the following sub-steps:
a1) reacting Naltrexone (II) and diethyl pyrocarbonate (DEPC) obtaining intermediate (V) and subsequently two intermediates (VI) and (VII) according to the scheme:

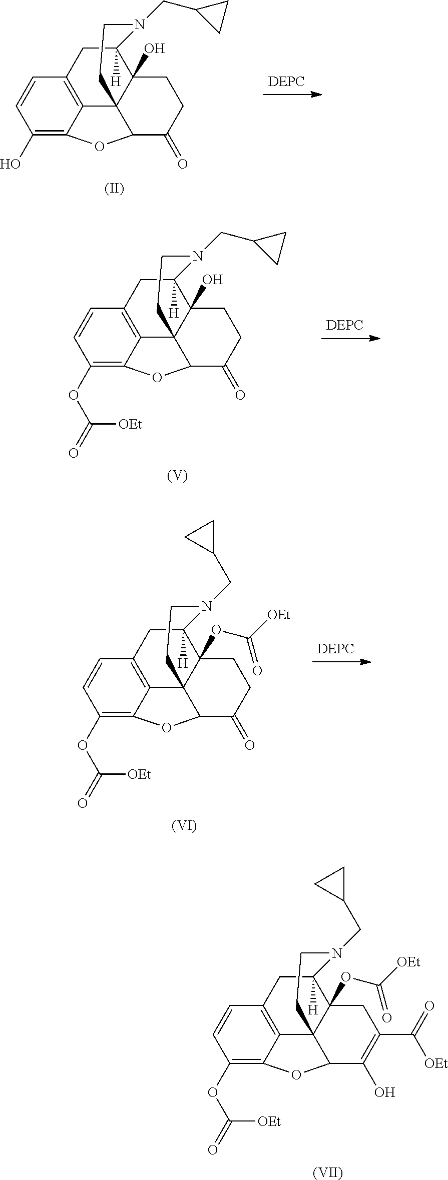

a2) treating intermediates (VI) and (VII) with a base to obtain compound (III)

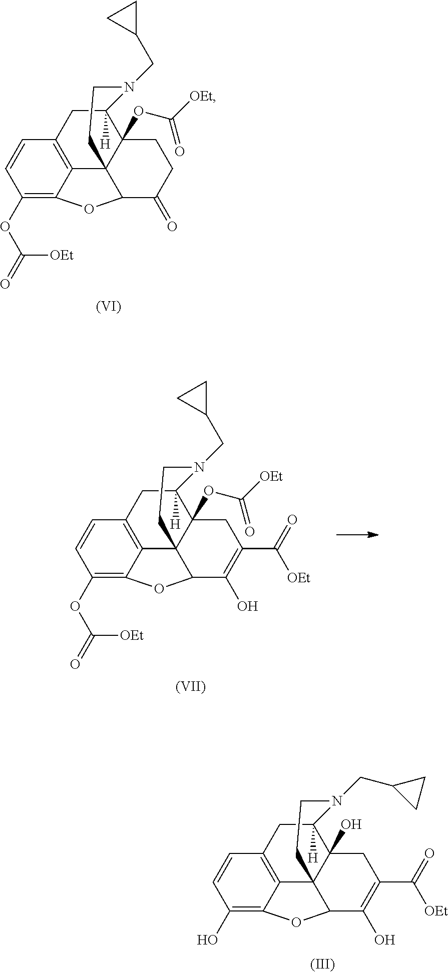

Intermediates (V), and specifically (VI) and (VII), are preferably not isolated and reaction a) takes place continuously.

Step a1) preferably takes place at a temperature in the range from 70° C. to 150° C., more preferably from 100° C. to 130° C., even more preferably under vacuum conditions.

In step a)/a1), diethyl pyrocarbonate is reacted in amounts of 2 to 12 equivalents, more preferably of 4 to 10 equivalents, even more preferably of 5 to 8 equivalents with respect to 1 equivalent of Naltrexone.

The treatment with the base of step a)/a2) preferably takes place at a temperature in the range from −20° C. to 40° C., more preferably from 0° C. to 20° C.

The base of step a/a2) is preferably selected from the group consisting of sodium ethoxide, potassium ethoxide, lithium hexamethyldisilazide, more preferably it is sodium ethoxide, more preferably as a solution in ethanol, even more preferably at 21% w/w in ethanol.

Said step a) preferably takes place in a reaction time ranging from 3 to 8 hours.

According to a preferred embodiment, the compound (III) obtained from step a), preferably in the form of solvate, is subjected to isolation and subsequent crystallization. Said isolation of compound (III) sub-step preferably takes place by adding to the reaction mixture of step a):—water or—an aqueous solution of a compound selected from the group consisting of sodium bicarbonate, potassium bicarbonate, sodium carbonate, potassium carbonate, sodium phosphate and potassium phosphate, more preferably potassium bicarbonate. The isolation of compound (III) therefore advantageously and preferably takes place by adding an aqueous solution of potassium bicarbonate to the reaction mixture of step a).

Said crystallization sub-step of the isolated compound (III) is preferably carried out with a solvent selected from the group consisting of ethanol, isopropanol, and butanol, preferably the solvent is ethanol.

According to the invention, step b) involves condensing compound (III) with compound (IV) with formation of the amide bond to obtain naldemedine (I).

Step b) preferably takes place at a temperature in the range from 110° C. to 180° C., more preferably from 150° C. to 170° C., even more preferably from 130° C. to 160° C. Said step b) preferably takes place under vacuum conditions.

The condensation step b) is carried out using from 1.0 to 4.0 equivalents of compound (IV), more preferably from 1.5 to 2.5 equivalents with respect to 1 equivalent of compound (III).

According to a preferred embodiment of the process of the invention, the reaction of step b) takes place in the absence of a solvent or in the presence of a suitable protic polar solvent. Preferably said suitable protic polar solvent is selected from the group consisting of ethanol, isopropanol, and butanol.

The reaction time of step b) is preferably from 2 to 5 hours, more preferably about 3 hours.

According to the present invention, the naldemedine (I) obtained from step b) can be obtained as a free base or as a salt.

Naldemedine obtained as a free base can advantageously be dissolved under heat in an organic solvent, for example benzene, toluene, xylene. Preferably said organic solvent is toluene.

Naldemedine in organic solution can therefore advantageously be extracted with a basic aqueous solution, more preferably this basic aqueous solution is selected from aqueous soda solution and aqueous potash solution.

Naldemedine in basic aqueous solution, preferably washed, can therefore advantageously be crystallized as a free base preferably by addition of an acid selected from the group consisting of p-toluenesulfonic acid, hydrochloric acid, and acetic acid, more preferably it is hydrochloric acid.

Naldemedine can then be subsequently salified by dissolution in a suitable organic solvent, preferably ethyl acetate, and salified by addition of an acid selected from the group consisting of p-toluenesulfonic acid, hydrochloric acid, and acetic acid, more preferably it is p-toluenesulfonic acid.

Said naldemedine salt is therefore preferably selected from the group consisting of p-toluenesulfonate, acetate, hydrochloride, preferably it is naldemedine para-toluenesulfonate.

### EXPERIMENTAL PART

The invention is now illustrated by some examples which are intended for illustrative and non-limiting purposes.

### EXAMPLES

### Example 1: Preparation of Compound (III)

Naltrexone hydrochloride (30 g, 1.0 eq), dichloromethane (500 mL, 10V) and a 2M aqueous solution of potassium bicarbonate (500 mL, 10V) were added into a reactor. The mixture was stirred vigorously, and the two phases were separated. The organic phase was dried with sodium sulfate and concentrated to dryness.

The progressive reaction of naltrexone with diethyl pyrocarbonate (DEPC) is shown in Reaction Scheme1:

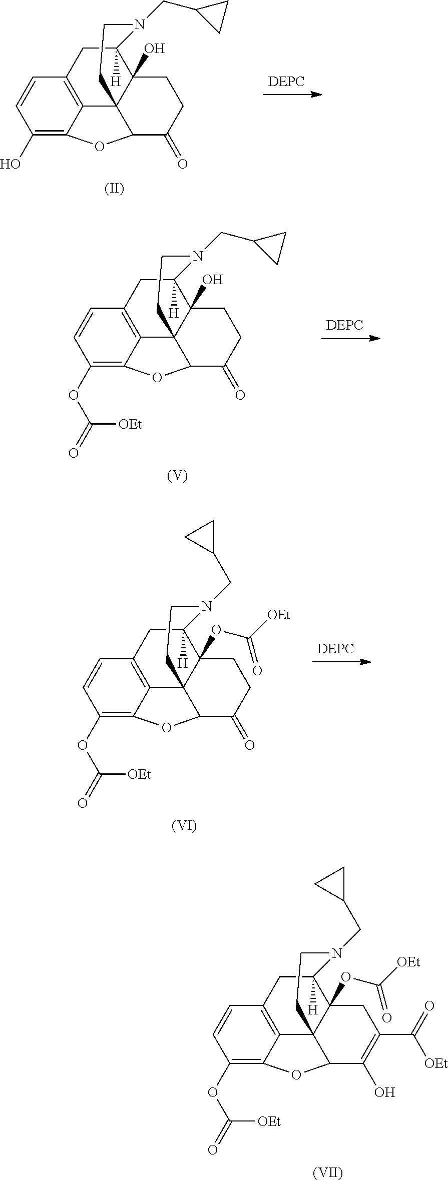

Diethyl pyrocarbonate (107.2 g, 8.0 eq) was added to the solid residue at room temperature and the mixture was stirred at room temperature until the solid was completely dissolved.

A solution sample was then taken and 1H-NMR and UPLC analysis with mass spectrometry were carried out (mass acquired: M+1).

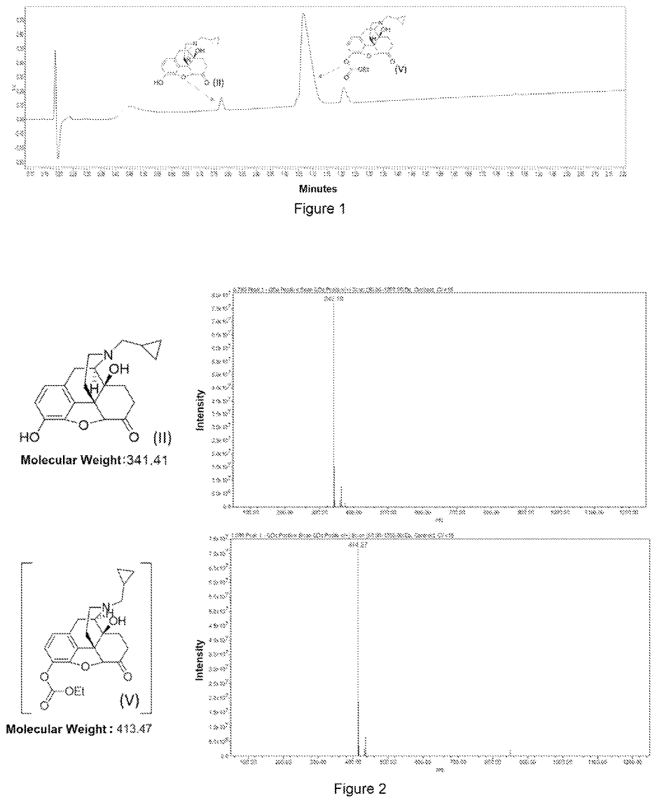

FIG. 1 reports the UPLC profile of the reaction mixture showing the peaks relating to Naltrexone (II) and to Naltrexone whose only phenolic group reacted with DEPC (intermediate V).

FIG. 2 shows, instead, the mass spectra of Naltrexone (II) and of Naltrexone whose only phenolic group reacted with DEPC (intermediate V).

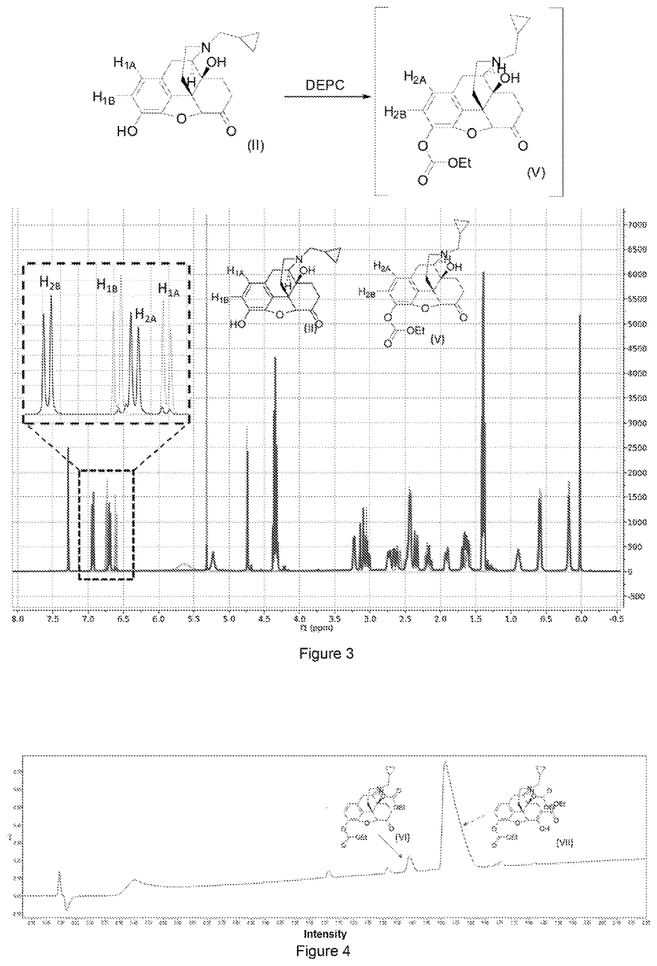

In addition to the mass spectrum, as further proof of intermediate (V) formation, the 1H-NMR spectra of Naltrexone (II) and intermediate (V) are shown in FIG. 3: as it can be seen, the only substantial difference between the two spectra is the one zoomed in the exploded view, in which it can be seen how the formation of the carbonate on the phenol of intermediate (V) shifts the doublets relating to the nearby aromatic protons towards more deshielded fields.

The solution was then heated to 130° C. under vacuum conditions and kept under stirring for 4 h.

A solution sample was then taken, and 1H-NMR and UPLC analysis with mass spectrometry were carried out (mass acquired: M+1).

FIG. 4 reports the UPLC profile of the reaction mixture showing the peaks relating to intermediate (VI) and intermediate (VII).

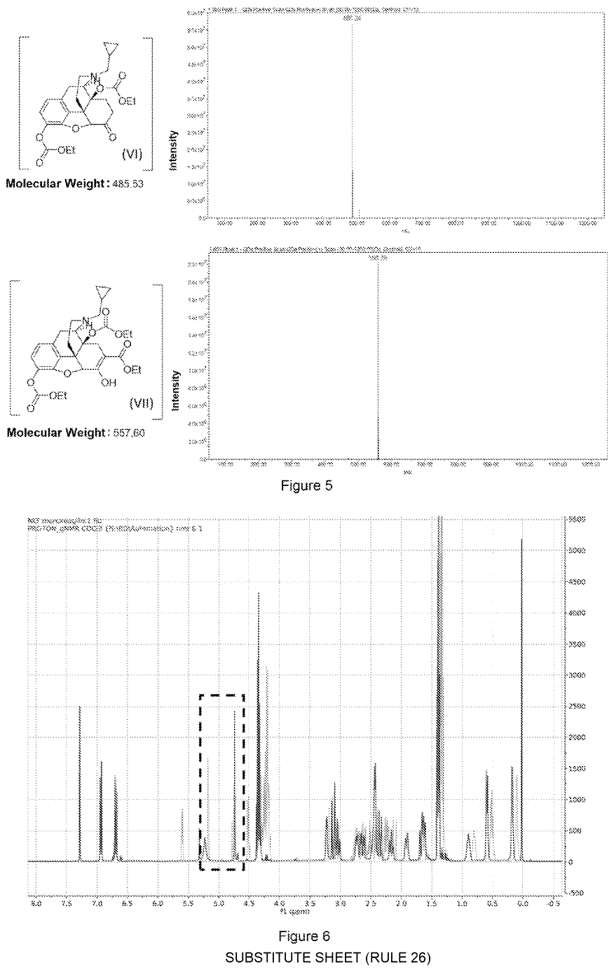

FIG. 5 shows, instead, the mass spectra of intermediate (VI) and intermediate (VII). In addition to the mass spectrum, as further proof of intermediates (VI) and (VII) formation, a 1H-NMR spectrum of the reaction mixture was carried out, which is shown in FIG. 6 superimposed with the 1H-NMR spectrum of intermediate (V): the darker gray spectrum refers to intermediate (V), while the lighter shade spectrum refers to a 20/80 mixture of intermediates (VI) and (VII).

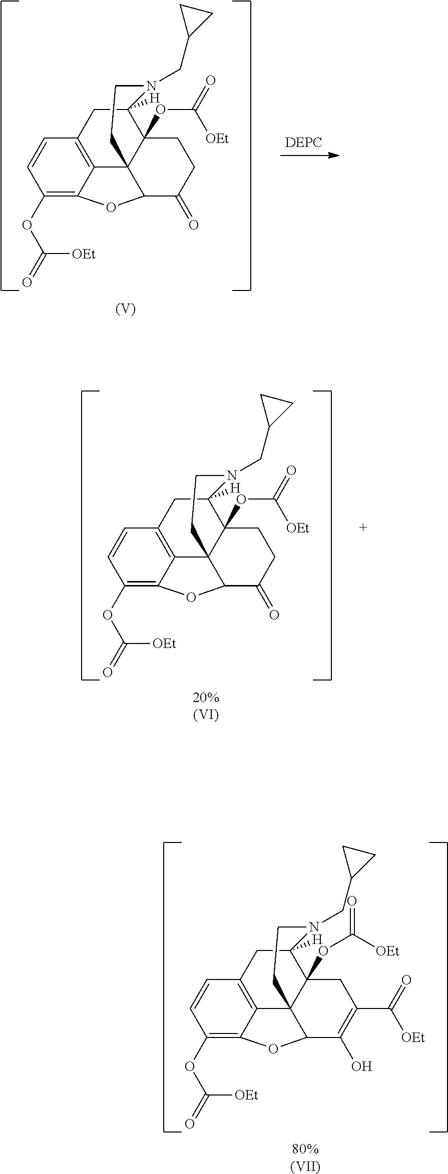

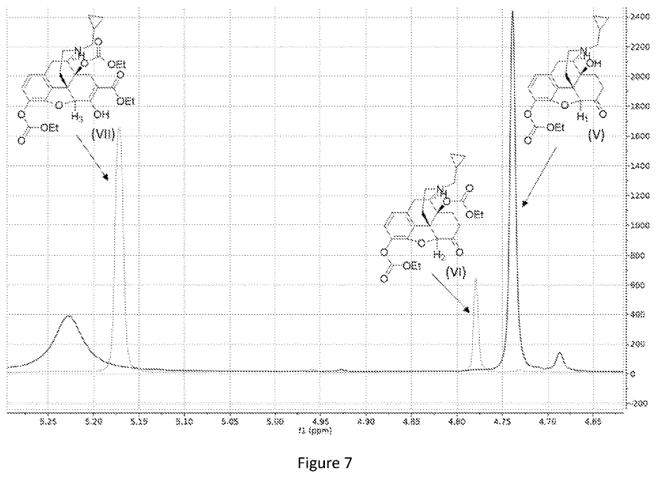

The spectrum area highlighted by a dotted line in FIG. 6 is exploded for easier understanding in FIG. 7.

As it can be seen in FIG. 7, the carbonate formation on the alcohol does not significantly affect the shielding of the proton of intermediate (VI) with respect to the corresponding one of intermediate (V), the two signals of the protons H₁ and H₂ are in fact very close, while, on the other hand, the formation of intermediate (VII) involves the presence of an alpha hydroxyl group on the proton H₃, with its consequent shift to higher fields.

Once the reaction end was confirmed, the reaction mixture was cooled to 70° C. and diluted in ethanol (30 mL, 1V). The reaction mixture was then cooled to 0° C., and sodium ethoxide (21% w/w solution in ethanol, 54.2 g, 2.0 eq) was slowly added to it, while maintaining the temperature between 0° C. and 10° C.; then, the mixture was left under stirring at 0° C. for 2 h.

After the end of the reaction was confirmed by UPLC, a 2M aqueous potassium bicarbonate solution (180 mL, 6V) was slowly added to the reaction mixture. After filtering the resulting solid, the latter was dissolved under heat in ethanol (240 mL, 8V) and concentrated under heat to a volume of 180 mL (6V). The solution was then slowly cooled, initially to room temperature, then, again slowly, to −20° C. The solid was then filtered and dried under vacuum to obtain compound (III) as an ethanol solvate (26.0 g, purity 99.5%, yield: 76%).

### Example 2: Preparation of Naldemedine FREE Base (I)

Compound (III) (20 g, 1.0 eq) and compound (IV) (17.6 g, 2.0 eq) were loaded into a reactor. The reaction mixture was heated to 150° C. for 3 h under vacuum conditions. After the end of the reaction was confirmed by UPLC, boiling toluene (about 108° C.) was added to the mixture (100 mL, 5V). The obtained solution was then cooled to room temperature and the product was extracted into 1 M NaOH (100 mL, 5V). The aqueous phase was washed twice with dichloromethane (100 mL, 5V), then the aqueous phase was acidified to pH 8 by slow addition of 1M HCl. The solid thus obtained was filtered and dried under vacuum at 50° C., and used in the subsequent step without further purification (17.5 g, purity 97.6%, yield 72%)

### Example 3: Preparation of Naldemedine p-Toluenesulfonic Salt

Naldemedine (10 g, 1 eq) was loaded into a reactor as a free base, which was dissolved in ethyl acetate (250 mL, 25V). The suspension thus obtained was filtered and the solution concentrated to a final volume of 150 mL (15V). Separately, a solution of p-toluenesulfonic acid monohydrate (4.0 g, 1.2 eq) in ethanol (20 mL, 2V) was prepared. The acid in ethanol solution was slowly added under heat (45° C.) to the ethyl acetate solution. At the end of the addition, the mixture was cooled to room temperature and the product was isolated by filtration. The solid thus obtained was dissolved under heat in ethanol (400 mL, 40V) and the solution was concentrated under heat to 100 mL (10V). The solution was then slowly cooled to room temperature. The solid was filtered and dried under vacuum to obtain naldemedine salt of p-toluenesulfonic acid (11.5 g, purity 99.9%, yield 90%).

1. A process for the preparation of naldemedine or a salt thereof, comprising the steps of:
a) reacting Naltrexone (II) and diethyl pyrocarbonate (DEPC) and treating with a base to give compound (III): 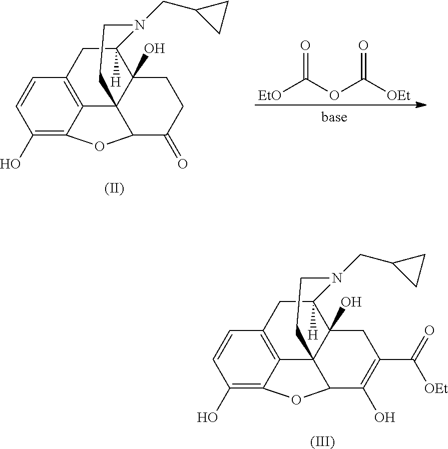
b) condensing compound (III) with compound (IV) with formation of the amide bond to obtain naldemedine (I):

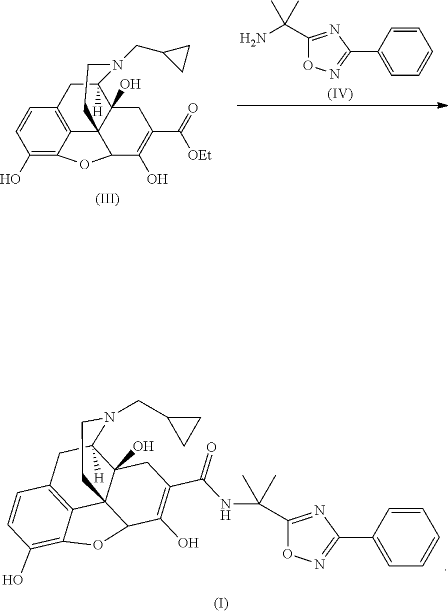

2. The process of claim 1, wherein step a) comprises the following sub-steps:
a1) reacting Naltrexone (II) and diethyl pyrocarbonate (DEPC) obtaining intermediate (V) and subsequently two intermediates (VI) and (VII) according to the scheme: 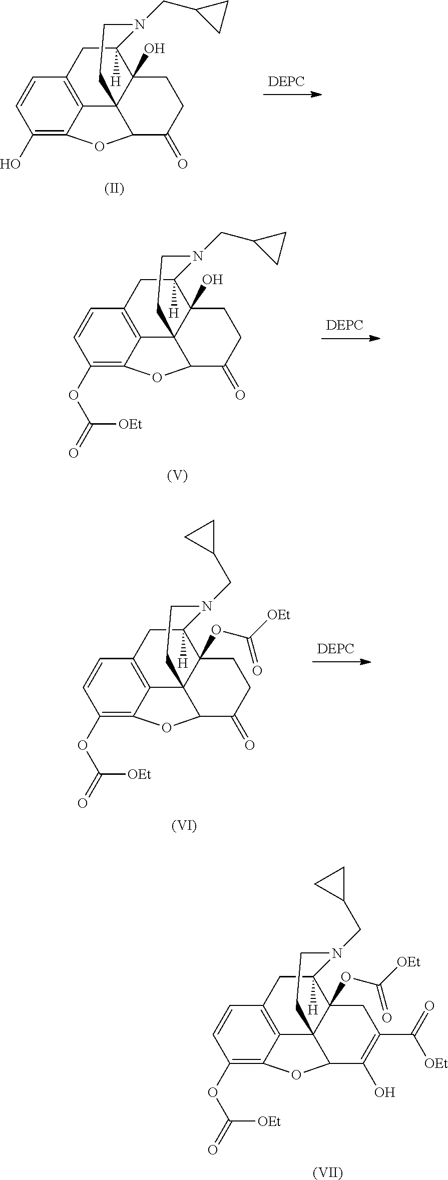
a2) treating intermediates (VI) and (VII) with a base to obtain compound (III)

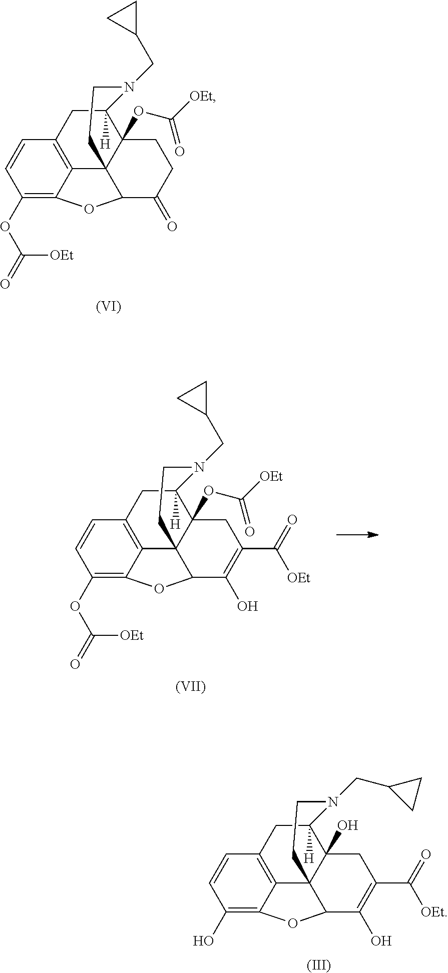

3. The process of claim 2, wherein intermediates (V), (VI) and (VII) are not isolated and the reaction a) takes place continuously.

4. The process of claim 1, wherein in step a)/a1) the reaction between Naltrexone (II) and diethyl pyrocarbonate (DEPC) takes place at a temperature in the range from 70° C. to 150° C.

5. The process according to claim 4, wherein in step a)/a1) the reaction between Naltrexone (II) and diethyl pyrocarbonate (DEPC) takes place at a temperature in the range from 100° C. to 130° C.

6. The process according to claim 1, wherein in step a)/a1) the reaction between Naltrexone (II) and diethyl pyrocarbonate (DEPC) takes place under vacuum conditions.

7. The process according to claim 1, wherein diethyl pyrocarbonate is reacted in amounts of 2 to 12 equivalents with respect to 1 equivalent of Naltrexone.

8. The process according to claim 7, wherein diethyl pyrocarbonate is reacted in amounts of 4 to 10 equivalents with respect to 1 equivalent of Naltrexone.

9. The process according to claim 8, wherein diethyl pyrocarbonate is reacted in amounts of 5 to 8 equivalents with respect to 1 equivalent of Naltrexone.

10. The process of claim 1, wherein in step a)/a2) the treatment with the base takes place at a temperature in the range from −20° C. to 40° C.

11. The process according to claim 10, wherein in step a)/a2) the treatment with the base takes place at a temperature in the range from 0° C. to 20° C.

12. The process of claim 1, wherein the base of step a/a2) is selected from the group consisting of sodium ethoxide, potassium ethoxide, lithium hexamethyldisilazide.

13. The process according to claim 12, wherein the base of step a/a2 is sodium ethoxide.

14. The process according to claim 13, wherein the base of step a/a2 is sodium ethoxide as a solution in ethanol, preferably at 21% w/w in ethanol.

15. The process of claim 1, wherein the compound (III) obtained from step a), preferably in the form of a solvate, is subjected to isolation and subsequent crystallization.

16. The process of claim 1, wherein step b) takes place at a temperature in the range from 110° C. to 180° C.

17. The process according to claim 16, wherein step b) takes place at a temperature in the range preferably from 150° C. to 170° C.

18. The process according to claim 17, wherein step b) takes place at a temperature in the range preferably from 130° C. to 160° C.

19. The process of claim 1, wherein step b) takes place under vacuum conditions.

20. The process of claim 1, wherein the condensation step b) is carried out using from 1.0 to 4.0 equivalents of compound (IV) with respect to 1 equivalent of compound (III).

21. The process according to claim 20, wherein the condensation step b) is carried out using from 1.5 to 2.5 equivalents with respect to 1 equivalent of compound (III).

22. The process of claim 1, wherein the reaction of step b) takes place in the absence of solvent or in the presence of a suitable protic polar solvent.

23. The process according to claim 22, wherein said suitable protic polar solvent is selected from the group consisting of ethanol, isopropanol, and butanol.

24. The process of claim 1, wherein the reaction time of step b) is from 2 to 5 hours.

25. The process according to claim 24, wherein the reaction time of step b) is about 3 hours.

26. The process of claim 1, wherein naldemedine (I) obtained from step b) can be obtained as a free base or as a salt.

27. The process of claim 1, wherein naldemedine (I) obtained as a free base is dissolved under heat in an organic solvent.

28. The process according to claim 27, wherein said organic solvent is toluene or xylene.

29. The process of claim 27, wherein naldemedine (I) in organic solution is extracted with a basic aqueous solution.

30. The process of claim 29, wherein such basic aqueous solution is selected from aqueous solution of caustic soda and caustic potash.

31. The process of claim 1, wherein naldemedine (I) is crystallized as a free base by addition of an acid selected from the group consisting of p-toluenesulfonic acid, hydrochloric acid, and acetic acid.

32. The process according to claim 31, wherein the acid is hydrochloric acid.
